(19) United States
(12) Patent Application Publication
(10) Pub. No.: US 2007/0073312 A1
(43) Pub. Date: Mar. 29, 2007

(54) Surgical instrument

(75) Inventors: Perry Robin Mykleby, Collierville, TN (US); Kevin Cooper Edwards, Olive Branch, MS (US); Phillip Andrew Ryan, Memphis, TN (US)

Correspondence Address: OLIFF & BERRIDGE, PLC, P.O. BOX 19928, ALEXANDRIA, VA, 22320, US

(73) Assignee: GYRUS ENT, L.L.C., Bartlett, TN (US)

(21) Appl. No.: 11/503,929

(22) Filed: Aug. 15, 2006

Related U.S. Application Data
(60) Provisional application No. 60/719,593, filed on Sep. 23, 2005.

(51) Int. Cl. A61B 17/26

(52) U.S. Cl. 606/113

(57) ABSTRACT

A surgical instrument includes an elongate shaft (3) adapted to be inserted within the human body, the elongate shaft (3) being formed of a shape memory material having an initial shape, the shape memory material being malleable at room temperature so that its shape can be changed by hand, and having a transition temperature such that it reverts to the initial shape when subject to a temperature rise associated with an autoclave sterilisation process.

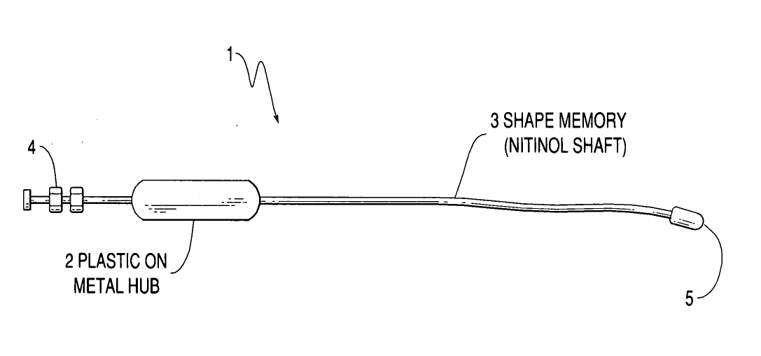

This invention relates to a surgical instrument, typically for use in minimally invasive or “keyhole” surgery.

Depending on the procedure to be performed, and the distinct physical constraints that vary from one patient to another, a surgical instrument often needs to be bent to a particular shape for each procedure. The present invention provides an improved instrument, taking into account the need for this repeated bending.

Accordingly, there is provided a surgical instrument including an elongate shaft adapted to be inserted within the human body, the elongate shaft being formed of a shape memory material having an initial shape, the shape memory material being malleable at room temperature so that its shape can be changed by hand, and having a transition temperature such that it reverts to the initial shape when subject to a temperature rise associated with a sterilization process.

The improved surgical instrument is thereby capable of being bent to shape during an individual procedure, and yet able to reset itself ready for the next procedure during the sterilisation process. The heat used during autoclaving causes the shape memory material to resume its initial shape ready for the next operation.

The shape memory material is preferably a shape memory metal such as Nitinol. Nitinol is a nickel-titanium alloy commonly used in military, medical safety and robotics applications. There are examples of shape memory materials being used in medical implants and prostheses, and in surgical instruments such as surgical cutting and retrieval instruments. Typically the instrument is inserted in one orientation, and deflects to a different shape in order to grasp and retrieve an object from within the body, such as kidney stones. Although the use of shape memory materials is known for surgical applications, it is generally considered for use in attaining different positions within the body. It has not been considered for use in resetting the shape of a surgical instrument after use, using the heat available from the sterilization of a reusable surgical instrument.

Other shape memory materials are known in the art including Copper-aluminum-nickel alloys, copper-zinc-aluminum alloys, and iron-manganese-silicon alloys, although the choice of shape memory material may depend on the biocompatibility of the material concerned as well as its shape memory properties.

In one convenient arrangement the shaft of the surgical instrument is straight in its initial shape. The surgeon can bend the straight shaft during use, and when the instrument is sterilized after use the shaft of the instrument will revert to its straight shape. In an alternative arrangement, the shaft of the surgical instrument is angled, having first and second straight portions with a curved section therebetween. In this arrangement, the surgeon can alter the radius of the curved portion during use, to provide an instrument with a range of angles. The sterilization of the instrument will cause the instrument to revert to its initial shape, ready for the next procedure. Finally, the shaft of the surgical instrument is conveniently a curved shaft, with a substantial section of the shaft being constituted by a steadily curving portion. In this arrangement, the surgeon can increase or decrease the curve of the instrument, or even straighten it completely, only to have it reset to its original position when the instrument is sterilized ready for re-use.

The surgical instrument is typically a tissue cutting or manipulating instrument, or alternatively may be a “support” instrument such as a suction tube. In one convenient embodiment of the invention, the instrument is a suction tube and has a lumen extending the entire length of the shaft.

The invention will now be further described, by way of example only, with reference to the accompanying drawing, which is a schematic representation of a suction tube in accordance with the invention.

Referring to the FIGURE, a suction tube is shown generally at 1, and comprises a plastics hub 2 and an elongate shaft 3 extending from the hub. A connector portion 4 is provided at the proximal end of the instrument, attached to the hub 2 opposite the shaft 3. A lumen (not shown) extends from the connector portion 4, through the hub 2, and extends the entire length of the shaft 3. The lumen exits the shaft at the distal tip thereof, by way of exit aperture 5.

The shaft 3 is formed of shape memory metal (Nitinol), and the shaft is initially a straight shaft. However, the shaft 3 is sufficiently thin such that it can readily be re-shaped by hand. At room temperature, the Nitinol is in its Martensite form, and will remain in whatever distorted shape is selected. Thus the surgeon is able to shape the shaft of the suction tube to whatever shape is required for the procedure. The connector portion 4 is attached to a source of suction, and material, including tissue, fluids, and other surgical debris, is evacuated through the shaft from the surgical site.

Following the procedure the suction tube is sent for autoclave sterilization. The heat applied to the suction tube during the sterilization process raises the temperature of the suction tube above the transition temperature of the shape memory material. This causes the shape memory material to transition to its Austenite form, and causes the shaft 3 to revert to its initial straight shape. Thus the suction tube 1 is ready for the next procedure, with the shaft in its initial straight shape without the surgeon or support staff needing to manually straighten the shaft.

The present invention is applicable to many different surgical instruments, and can be used with instruments having a variety of initial configurations. Whatever, the initial configuration, any changes to the shape of the instrument applied by the surgeon or caused during the surgical procedure, can be removed during the sterilization process, with the shaft of the instrument reverting to its original condition.

1. A surgical instrument including an elongate shaft adapted to be inserted within the human body, the elongate shaft being formed of a shape memory material having an initial shape, the shape memory material being malleable at room temperature so that its shape can be changed by hand, and having a transition temperature such that it reverts to the initial shape when subject to a temperature rise associated with a sterilisation process.

2. A surgical instrument according to claim 1 wherein the shape memory material is a shape memory metal.

3. A surgical instrument according to claim 2 wherein the shape memory metal is Nitinol.

4. A surgical instrument according to claim 1 wherein the shaft is a straight shaft when in its initial shape.

5. A surgical instrument according to claim 1 wherein the shaft is an angled shaft when in its initial shape, having first and second straight portions separated by a curved section.

6. A surgical instrument according to claim 1 wherein the shaft is a curved shaft when in its initial shape, a substantial section of the shaft being constituted by a steadily curving portion.

7. A surgical instrument according to claim 1 wherein the shaft has a lumen extending the entire length of the shaft.

8. A surgical instrument according to claim 7 wherein the instrument is a suction tube.
